(19) United States
(12) Patent Application Publication
(10) Pub. No.: US 2022/0244278 A1
(43) Pub. Date: Aug. 4, 2022

(54) NANO-PROBE FOR MEASURING pH IN SINGLE CELLS, AND METHOD AND APPARATUS FOR MEASURING pH IN SINGLE CELLS USING THE SAME

(71) Applicant: POSTECH Research and Business Development Foundation, Pohang-si (KR)

(72) Inventors: Jung Ho JE, Pohang-si (KR); Seung Soo OH, Pohang-si (KR); Moon Jung YONG, Incheon (KR); Un YANG, Pohang-si (KR); Byung Hwa KANG, Pohang-si (KR)

(21) Appl. No.: 17/588,571

(22) Filed: Jan. 31, 2022

(30) Foreign Application Priority Data
Feb. 1, 2021 (KR) 10-2021-0014230
Jan. 28, 2022 (KR) 10-2022-0013182

(51) Int. Cl. G01N 33/84; G01N 21/64

(52) U.S. Cl. CPC G01N 33/84; G01N 2021/7786; G01N 2021/6439; G01N 21/6458

(57) ABSTRACT

Provided is a method and apparatus for measuring pH in single cells, and a method of manufacturing a nanoprobe therefor. The apparatus for measuring pH in a single cell comprises: a nanoprobe formed by labeling a pH-responsive fluorescent material to a nanowire grown on a tapered tip of an optical fiber; a manipulator capable of regulating a three-dimensional movement of the nanoprobe to insert the nanoprobe into a single living cell; a light source for applying light to the optical fiber; an optical coupler for connecting the optical fiber with another optical fiber to transmit the light incident through the optical fiber to the nanoprobe and to transmit a fluorescence signal obtained from the nanoprobe through the another optical fiber; and a spectrometer for obtaining a pH value by receiving the fluorescence signal through the another optical fiber and analyzing spectral data from the fluorescence signal.

### FIELD OF THE INVENTION

The present invention relates to a pH monitoring in single cells, and more particularly, to a nano-probe for accurately measuring pH in single cells, and a method and apparatus for measuring pH in single cells using the same.

### BACKGROUND

Conventionally, all cells were considered to be homogeneous and were analyzed collectively, but it was recently found that individual cells are actually heterogeneous (see Cell Cycle 12, 3640-3649 (2013)). Accordingly, in recent years, a technique for analyzing individual characteristics of a single cell has been attracting attention (see Nat. Cell Biol. 20, 1349-1360 (2018)).

Factors related to cell characteristics such as pH, mRNA, and protein are various. Among them, pH is an important factor because it affects intracellular protein metabolism and directly affects cell function (see J. Immunol. Methods. 221, 43-57 (1998)). It is also known that intracellular pH measurement is used as a standard for diagnosing diseases such as cancer (see Biochemistry. 35, 2811-2817 (1996)).

In particular, as the nucleus of a cancer cell divides rapidly, it is expected that the pH of the cancer cell is different from that of normal cells (see Chem. Soc. Rev. 46, 3830-3852 (2017); and Nat. Rev. Cancer. 11, 671677 (2011)). However, it is known that to measure the pH inside a nucleus is very difficult because the nucleus is not only deep in the cytosol but also surrounded by a nuclear membrane. Therefore, the technique of measuring pH in a single cell nucleus is more difficult than that of measuring pH of a single cytosol.

Accordingly, in the prior art, the following methods were used to measure pH in a single cell.

First, a nanoparticle insertion-based technology for measuring intracellular pH involves inserting fluorescent nanomaterial that responds to pH, and then analyzing a signal at the outside of the cell (see J. Am. Chem. Soc. 136, 12253-12256 (2014); Anal. Chem. 91, 8383-8389 (2019); and Analyst 145, 5768-5775 (2020)). However, this method is impossible for analyzing the pH of cells in their natural state due to the insertion of foreign substances into cells, which causes cell contamination. Furthermore, the accuracy of this method is low owing to randomness of inserting fluorescent nanomaterials into single cells and the light scattering during the pass of the signal light from inside the cell to outside the cell. Moreover, to measure the pH of the nucleus in this method is known to be very difficult because it is almost impossible to insert nanomaterials into the nucleus of a single cell.

Another method is a probe insertion-based method, which involves measuring intracellular pH by inserting a probe including a substance that responds to pH in the cell (see Sensors Actuators, B Chem. 290, 527-534 (2019); and Analyst 145, 4852-4859 (2020)). In this method, a probe is prepared by conjugating a pH-reactive material to the surface of a tapered glass capillary. However, because the diameter of the probe gradually becomes thicker from the probe tip, the insertion of the probe inside a desired position in the cell can cause cell damage. Furthermore, since light is irradiated from the outside of the cell and the reflected light is received from the outside of the cell, severe scattering of light is unavoidable in the process of the light passing through various media, resulting in poor accuracy. At this time, the pH in the cell is measured by obtaining the ‘spectrum generated by the pH-reactive material’.

### SUMMARY OF THE INVENTION

Accordingly, the present invention has been developed to solve the above problems, and it is the object of the invention to provide a nanoprobe capable of accurately measuring pH in a single living cell in real time without contamination or damage to the cell when the nanoprobe is inserted into the cell and a method for measuring pH in a single cell using the same and an apparatus thereof.

According to one aspect of the present invention for achieving the above object, the method of measuring pH in a single cell comprises: (a) inserting a nanoprobe into the single cell, wherein the nanoprobe is prepared by labeling a pH responsive fluorescent material to the surface of a nanowire grown on a tapered tip of an optical fiber; (b) injecting a light through the optical fiber into the nanoprobe; (c) exciting the pH-responsive fluorescent material by the light to generate fluorescence; (d) transmitting the fluorescence signal generated from the fluorescence material according to pH in the cell, through the optical fiber; and (e) analyzing the fluorescence signal to obtain a pH value in the cell.

The method of manufacturing a nanoprobe according to the present invention comprises: (a) filling a nanopipette with a nanowire material solution and pulling down the nanopipette to bring the nanowire material solution into contact with the tip of an optical fiber; (b) pulling up the nanopipette to grow a nanowire on a tip of the optical fiber; (c) filling a micropipette with an aqueous solution containing a pH-responsive fluorescent material and pulling down the micropipette to immerse a part of the nanowire in the aqueous solution; and (d) pulling up the micropipette to form a nanoprobe labeled with a pH-responsive fluorescent material.

According to another aspect of the present invention, a nanoprobe for measuring pH in a single cell comprises: an optical fiber; a nanowire formed by growing a nanowire material solution at one end of the optical fiber; and a pH-responsive fluorescent material labeled on a part of the nanowire.

In the present invention, the nanowire material solution is a hydrophobic polymer solution, and the hydrophobic polymer solution may be selected from the group consisting of at least PVBN₃, PVB-alkyne, and PVB-COOH. In addition, the optical fiber has a tapered tip, and the pH-responsive fluorescent material is a fluorescein molecule having a functional group capable of being conjugated to the nanowire, wherein the fluorescein may be selected from the group consisting of at least DBCO-FAM, Azide-FAM, and Amine-FAM. According to the present invention, the wetting (or labeled) length of the nanowire by the pH-responsive fluorescent material is controlled to be 100 nm to 900 nm, preferably 500 nm or less.

According to another aspect of the present invention, an apparatus for measuring pH in a single cell comprises: a nanoprobe formed by labeling a pH-responsive fluorescent material to a nanowire grown on a tapered tip of an optical fiber; a manipulator capable of regulating a three-dimensional movement of the nanoprobe so as to insert the nanoprobe into a single living cell; a light source for applying light to the optical fiber; an optical coupler for connecting the optical fiber with another optical fiber so as to transmit the light incident through the optical fiber to the nanoprobe and so as to transmit a fluorescence signal obtained from the nanoprobe through the another optical fiber; and a spectrometer for obtaining a pH value by receiving the fluorescence signal through the another optical fiber and analyzing spectral data from the fluorescence signal.

In the present embodiment, the nanoprobe may have a uniform diameter. The nanoprobe has a diameter of 10 nm to 900 nm, and preferably 400 nm or less, and has a length of 1 μm to 10 μm, and preferably 5 μm or less.

In the present embodiment, the light incident through the optical fiber may be near infrared or visible light, and the light may have a wavelength of 300 nm to 1000 nm, and preferably 400 nm to 700 nm. The wavelength of light incident through the optical fiber is selectable according to the component, shape and optical properties of the nanoprobe, the type of the target molecule to be detected, and the type of the target cell.

According to another aspect of the present invention, a method of preparing a nanowire material solution comprises steps of: mixing a mixture of PVC (0.014 g, 131 mmol) and sodium azide (0.010 g, 220 mmol) in anhydrous DMF solvent (0.7 mL) in an amber vial at 70° C. and then covering the vial with an aluminum foil to block light; adding methanol (0.5 mL) to the mixed solution after 2 hours of reaction, and centrifuging the same at 10,000 rpm for 1 minute to remove an excess unreacted reagent and precipitate an azide-functionalized polymer; and drying the obtained precipitates in a vacuum condition for 1 hour and then dissolving the precipitates by adding an NMP solvent (50 μL).

It is important to understand cellular heterogeneity and metabolism through local pH monitoring. Therefore, monitoring the spatiotemporal pH of single living cells beyond cell and organelle membranes is challenging.

In the present invention, the inventors have developed a nanoprobe with high mechanical strength that enables in situ monitoring of pH dynamics in desired organelles through direct optical communication. By chemically labelling fluorescein at one end of a polyvinylbenzyl azide nanowire, the inventors continuously monitored pH variations of different compartments inside a living cell, successfully observing pH homeostasis and stimuli-selective pH variations of specific organelles.

Importantly, the inventors demonstrated for the first time that during the human cell cycle, the nucleus displays pH homeostasis in interphase but pH variation in the mitotic phase, thereby participating in independent pH regulation by the nuclear membrane. The rapid and accurate local pH detection and reporting capability of the nanoprobe would be highly valuable for investigating cellular behaviours under diverse biological situations in various living cells.

Meanwhile, according to the above-described features, the present invention provides the following effects.

1) The device for measuring pH in a single cell using the nanoprobe according to the present invention allows the accurate measurement of pH for each position inside the single cell without contamination or damage to the cell when inserted into the cell.

2) The device for measuring pH according to the present invention allows measuring the change in pH according to time or environment change in a single cell in real time without contamination or damage to the cell when inserted into the cell.

3) The pH measuring device according to the present invention allows accurately measuring pH of the cytosol and the cell nucleus of a single cell without contamination and damage to the cell when inserted into the cell, and also accurately measuring pH in other organelles in the cell can also be accurately measured.

4) The pH measuring device according to the present invention allows measuring pH change in the nucleus during the growth of a single cell in real time.

### BRIEF DESCRIPTION OF THE DRAWINGS

FIG. 1 is a view showing a device configuration design of a nanoprobe (i.e., nanowire waveguide) for detecting and transmitting spatiotemporal pH changes within a single living cell (a), an electron microscopy image of the nanoprobe (b), and a pH monitoring in a single living cell using the nanoprobe (c);

FIG. 2 is graphs showing the ¹H-NMR spectra of PVC (Poly(vinylbenzyl chloride)) and PVBN₃ (Poly(vinylbenzyl azide));

FIG. 3 is a view and photographs showing a fabrication procedure of a PVBN₃ nanowire on the tip of a tapered optical fiber;

FIG. 4 is photographs showing the conjugation process of DBCO-functionalized fluorescein (FAM) to a PVBN₃ nanowire;

FIG. 5 is photographs showing a mechanical property evaluation of the nanoprobe by inserting it into an agar gel;

FIG. 6 is photographs showing the optical loss evaluation at the junction between a nanowire and a tapered optical fiber;

FIG. 7 is graphs and photographs showing the optical response of the nanoprobe to the local pH variations;

FIG. 8 is graphs of the photostability and reproducibility of the nanoprobe;

FIG. 9 is photographs comparing the cell viability between the insertions of the nanoprobe and a tapered optical fiber into living HeLa cells;

FIG. 10 is a graph showing the histogram of cell viability after the extraction of the inserted nanoprobe (gray) and the tapered optical fiber (white) from cytosol and nucleus, respectively;

FIG. 11 is a view showing a Boltzmann fitting for obtaining a pH calibration curve targeting intracellular environments;

FIG. 12 is photographs and graphs showing the results of investigation for pH-dependent fluorescent signals of the nanoprobe at the outside and inside of living HeLa cells;

FIG. 13 is images showing the results of pH value monitoring in the cytosol and the nuclei during an entire cell cycle of single cells using a nanoprobe;

FIG. 14 is photographs of bright field and merged (bright field and fluorescence) images for the insertion of a nanoprobe into single living HeLa cells during mitotic phase, observed by confocal microscopy;

FIG. 15 is photographs of merged (bright field and fluorescence) images of HeLa cells, stained with nucleus-specific Hoechst dye (white), during mitotic phase (from prophase to cytokinesis) for pH measurement, observed by confocal microscopy;

FIG. 16 is a view and photograph showing cytosolic pH variations in response to external calcium ions;

FIG. 17 is photographs and graphs showing the real-time measurements of cytosolic pH of HeLa cells treated by excessive magnesium ion (5 mM); and

FIG. 18 is photographs showing a merged (bright field and fluorescence) and dark field images of living HeLa cells treated by excessive calcium ion (a) and excessive magnesium ion (b).

### DETAILED DESCRIPTION OF THE INVENTION

Hereinafter, preferred embodiments of the present invention will be described with reference to the accompanying drawings. In the following embodiments, portions excluding inevitable portions in the explanation of the invention, the illustration and explanation thereof are omitted, and the same reference numerals are assigned to the same or similar elements throughout the description and detailed explanation thereof will be omitted without repetition.

Cells are different from each other. Even in the same environments, genetically identical cells can display cell-to-cell variabilities, including cell morphology, proliferation, growth and survival rates, as a result of their own vital activities due to individual compartmentalization (see Stoeger, T., Battich, N. & Pelkmans, L. Passive Noise Filtering by Cellular Compartmentalization. Cell 164, 1151-1161 (2016)). To understand the different behaviours of individual cells, it is important to measure and analyse the changes in physiological parameters (e.g., pH, temperature, and oxygen levels) inside living cells (see Zhang, X. ai et al. Quadruply-labeled serum albumin as a biodegradable nanosensor for simultaneous fluorescence imaging of intracellular pH values, oxygen and temperature. Microchim. Acta 186, (2019)). In particular, organelles, such as the nucleus, mitochondria, endoplasmic reticulum, and Golgi apparatus, perform biological functions occasionally, and thus the changes in the different organelles should be independently monitored over time (see Jaworska, A., Malek, K. & Kudelski, A. Intracellular pH—Advantages and pitfalls of surface-enhanced Raman scattering and fluorescence microscopy—A review. Spectrochim. Acta—Part A Mol. Biomol. Spectrosc. 251, 119410 (2021); and Han, J. & Burgess, K. Fluorescent indicators for intracellular pH. Chem. Rev. 110, 2709-2728 (2010)). Specifically, due to different levels of cellular metabolism and homeostasis, there can be spatiotemporal pH heterogeneity according to the individual cells (see Sondergaard, R. V., Henriksen, J. R. & Andresen, T. L. Design, calibration and application of broad-range optical nanosensors for determining intracellular pH. Nat. Protoc. 9, 2841-2858 (2014)). Theoretically, local pH has been predicted to fluctuate differently during cell division by successive catabolism or anabolism processes, and when activated by apoptotic stimuli, programmed cell death leads to mitochondrial dysfunction, followed by abrupt acidification of the intracellular milieu (see Matsuyama, S., Llopis, J., Deveraux, Q. L., Tsien, R. Y. & Reed, J. C. Changes in intramitochondrial and cytosolic pH: Early events that modulate caspase activation during apoptosis. Nat. Cell Biol. 2, 318-325 (2000)).

Due to the significance of local pH variations, extensive studies has been conducted on the development of in situ monitoring systems capable of detecting and transmitting (or reporting) a subcellular pH in real time. A variety of pH-sensitive molecular probes (e.g., fluorescent dyes, quantum dots, and nanoparticles) are available for pH detection (see He, C., Lu, K. & Lin, W. Nanoscale metal-organic frameworks for real-time intracellular pH sensing in live cells. J. Am. Chem. Soc. 136, 12253-12256 (2014); Dennis, A. M., Rhee, W. J., Sotto, D., Dublin, S. N. & Bao, G. Quantum dot-fluorescent protein fret probes for sensing intracellular pH. ACS Nano 6, 2917-2924 (2012); and Shen, Y. et al. Organelle-targeting surface-enhanced Raman scattering (SERS) nanosensors for subcellular pH sensing. Nanoscale 10, 1622-1630 (2018)) that can be internalized into cells by electroporation or thorough endocytosis across otherwise impermeable cell membranes (see Albertazzi, L., Storti, B., Marchetti, L. & Beltram, F. Delivery and subcellular targeting of dendrimer-based fluorescent pH sensors in living cells. J. Am. Chem. Soc. 132, 18158-18167 (2010)). However, due to the nature of spontaneous internalization into cells, positioning the probes in a desired location, especially inside a membrane-protected organelle, remains a technical challenge. Although pH-responsive fluorescent proteins can be genetically encoded inside an engineered cell, elaborate gene engineering relevant to their expression, and subsequent transportation by protein trafficking is extremely difficult (see Green fluorescent protein as a noninvasive intracellular pH indicator. Biophys. J. 74, 1591-1599 (1998); and Palmer, A. E., Qin, Y., Park, J. G. & McCombs, J. E. Design and application of genetically encoded biosensors. Trends Biotechnol. 29, 144-152 (2011)). As an alternative, nanopipettes (see Zhang, Y. et al. Spearhead Nanometric Field-Effect Transistor Sensors for Single-Cell Analysis. ACS Nano 10, 3214-3221 (2016); and Guo, J. et al. Dynamic single-cell intracellular pH sensing using a SERS-active nanopipette. Analyst 145, 4852-4859 (2020)) or optical fibers (see Yang, Q. et al. Label-free in situ pH monitoring in a single living cell using an optical nanoprobe. Med. Devices Sensors 3, 1-10 (2020)) have been directly inserted into a target cell. However, without precise control of their size and shape, drilling a hole in the membrane is fatal to the cell. In addition, their pH detection could not be localized due to the difficulty in surface modifications and manipulations of the nanostructured materials, and the weak detection signals were frequently distorted by complex cellular environments (see Yan, R. et al. Nanowire-based single-cell endoscopy. Nat. Nanotechnol. 7, 191-196 (2011); and Lin, L. et al. Real-time tracing the changes in the intracellular pH value during apoptosis by near-infrared ratiometric fluorescence imaging. Chem. Commun. 54, 9071-9074 (2018)). Therefore, it is still necessary to develop a technology that enables real-time pH monitoring for each organelle in cells across multiple impermeable membranes in a single living cell.

In the present invention, the inventors fabricated a nanoprobe with high mechanical strength and sufficiently small diameter capable of monitoring pH dynamics in desired cellular compartments via direct optical communication. The polyvinylbenzyl azide (PVBN₃) nanowire according to the present invention is structurally strong and long enough to penetrate cell and organelle membranes, while its narrow diameter (˜200 nm) ensures negligible cell damage and leakage. Chemically labelled high-density fluorescein on the terminal of the nanoprobe can quickly respond to local pH variations, and through the nanoprobe, the pH-sensitive photoluminescence (PL) signals are directly transmitted to a spectrometer (<100 ms), minimizing optical loss and surrounding noise. Using the novel in situ pH detection system, the inventors continuously monitored pH changes of different compartments inside a single living cell, allowing several scientific discoveries, such as organelle-exclusive pH homeostasis and stimuli-selective pH regulations. In particular, the inventors demonstrated for the first time that during the cell cycle, the nucleus displays pH homeostasis at an interphase but pH fluctuation at a mitotic phase, newly implying the existence of independent pH-regulating activities by the nuclear envelope; this is attributed to the unique capability of the nanoprobe of the present invention in the live streaming of subcellular events by local pH monitoring of a single living cell.

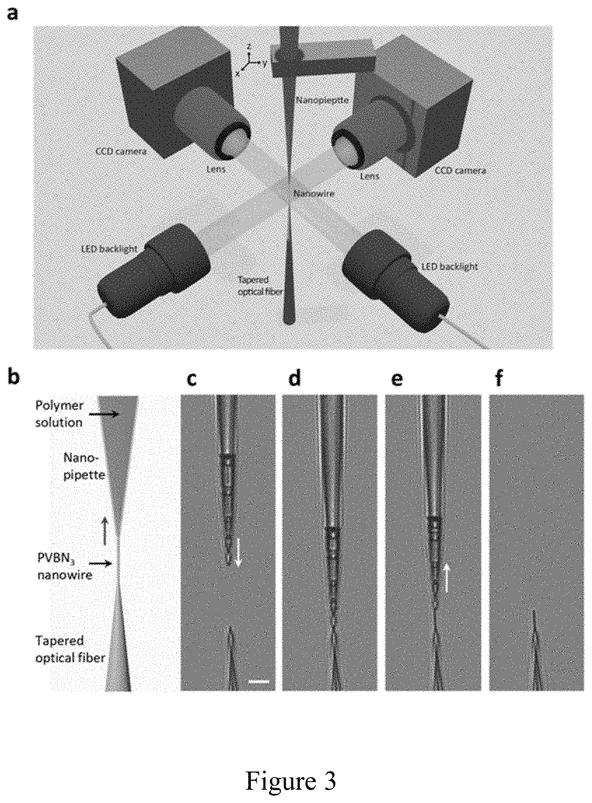

In the present invention, single intracellular pH measurement starts with fabricating a nanoprobe having a uniform diameter that can respond to pH by directly growing it on the tip of a tapered optical fiber. The nanoprobe includes a pH-responsive fluorescent material on its surface, and a method of fabricating the nanoprobe will be described with reference to FIG. 3 in the Examples below. For reference, here, ‘single cell’ means to include a single living cell and a single dead cell. In addition, ‘measurement of pH in a single cell’ means including measurement of pH not only in the nucleus of a cell, but also in the cytoplasm and in other intracellular organelles.

By the single intracellular pH measurement method of the present invention, it is possible to accurately measure pH inside a cell by inserting a nanoprobe into the cell and acquiring the fluorescence signal generated according to pH of the cell through an optical fiber and directly analyzing it.

In addition, by the single intracellular pH measurement method of the present invention, it is possible to measure the intracellular pH variation in real time by directly measuring the change in the fluorescence signal when pH of the cell changes according to time or environment.

In addition, by the single intracellular pH measurement method of the present invention, the diameter of the nanoprobe is sufficiently small and uniform, so there is almost no cell damage, and by receiving a signal directly from a desired position in the cell, it is possible to accurately measure pH at each intracellular location.

### Exemplary Embodiment

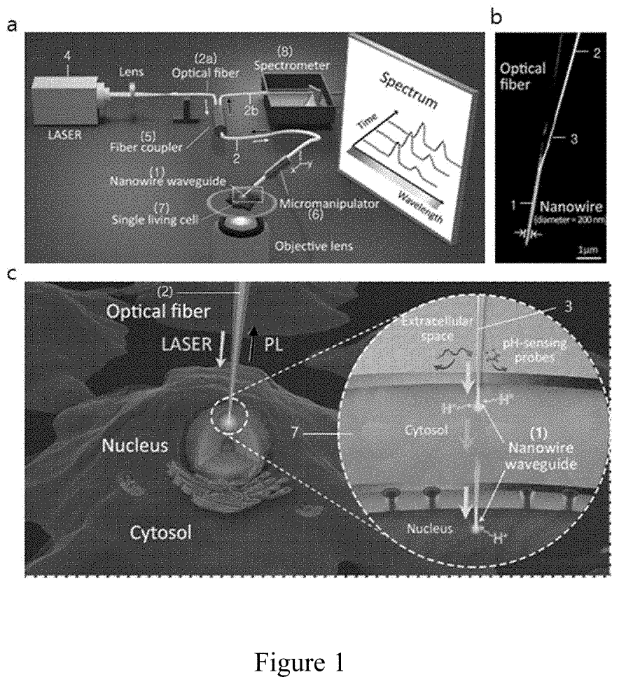

FIGS. 1a to 1c show the design configuration of a nanoprobe capable of detecting the change in spatiotemporal pH in a single living cell and pH detection in a single living cell using the nanoprobe. FIG. 1a shows an overall schematic view of a nanoprobe system for in situ pH monitoring of intracellular compartments in real time through direct optical communication.

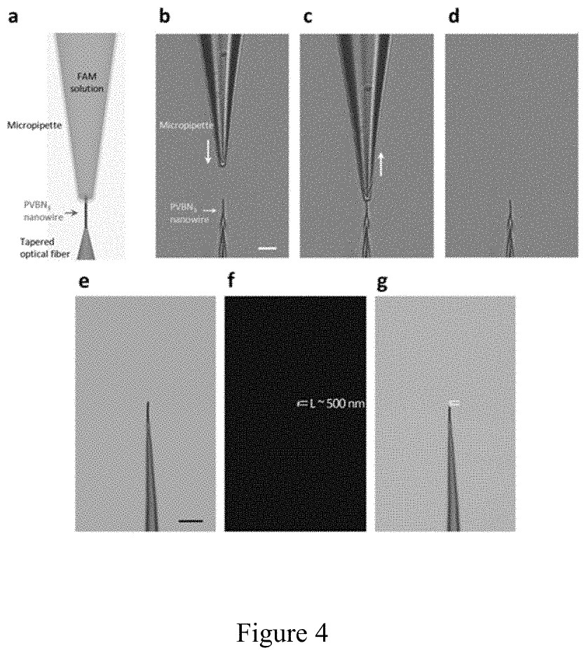

The nanoprobe (or “nanowire waveguide”; 1) is formed by conjugating a pH-responsive fluorescent material to a nanowire grown on a tapered tip 3 (FIG. 1b) of an optical fiber 2 (FIGS. 4a to 4g), wherein a laser incident from a light source (laser generator; 4) to a first optical fiber 2a reaches the nanoprobe 1 through the optical fiber 2 (white arrow).

In this embodiment, the optical fiber 2 is branched into the first optical fiber 2a for transmitting to the nanoprobe 1 a light (laser beam) incident from the light source 4 and a second optical fiber 2b for transmitting to a spectrometer 8 a fluorescence signal generated from the fluorescent material on the surface of the nanoprobe, and the first optical fiber 2a and second optical fiber 2b are combined into one body in a fiber coupler 5 leading to the nanoprobe 1. The optical coupler 5 guides the incident light from the first optical fiber 2a only to the nanoprobe 1 and transmit the fluorescence signal generated from the fluorescent material on the surface of the nanoprobe 1 only to the spectrometer 8.

The nanoprobe 1 is inserted into a single living cell 7 using a manipulator 6 having a micrometer resolution, capable of a three-dimensional movement control (see FIG. 1c). Positioning the nanoprobe within a living cell can be precisely controlled by a 3-axis micromanipulator 6 under observation by confocal fluorescence microscopy. At this time, a light (laser beam) reaching the nanoprobe 1 from the light source 4 through the first optical fiber 2a and the optical coupler 5 generates an evanescent wave, and by exciting pH responsive fluorescent material at the tip of the nanoprobe, the pH-responsive fluorescent material emits a photoluminescence (PL) signal. After this signal is transmitted to the optical fiber 2 through the nanoprobe 1, it is guided to the second optical fiber 2b by the optical coupler 5 without being subjected to environmental interference in optical communication, and directly transmitted to the spectrometer 8 (black arrow). Through this process, pH value is measured from spectral data of fluorescence obtained by the spectrometer 8. In this case, since the fluorescence signal of the nanoprobe 1 in the inside of the cell (FIG. 1c) is directly measured by the spectrometer 8 without distortion, it is possible to accurately measure pH value.

FIG. 1b shows a field emission scanning electron microscopy image (scale bar 1 μm) of a nanoprobe of the present invention grown directly on the tip of a tapered optical fiber, and FIG. 1c represents an image of local pH monitoring of living cells across the rigid membrane of a cell or organelle. A nanoprobe with a long length and a small diameter of the present invention is mechanically robust and does not induce cell leakage during membrane penetration, and the fluorescently labeled tip can easily reach a desired location (in the cytoplasm or nucleus) for in situ pH detection (see insertion drawing). Depending on the local intracellular proton concentration, the intensity of the PL signal changes rapidly, which can be monitored in real time through a nanoprobe.

In the present invention, the nanoprobe 1 is capable of penetrating not only the cell membrane but also the nuclear membrane, so it is possible to measure pH in the nucleus as well as the cytoplasm (see FIG. 1c; of course, it is also possible to measure pH of other intracellular organelles). In addition, since the nanoprobe 1 has a sufficiently small diameter (d: 10 nm to 900 nm or less, preferably 400 nm or less), there is an advantage of no or negligible cell damage. In addition, since the nanoprobe 1 has a sufficiently short length (l: 1 μm to 10 μm or less, preferably 5 μm or less), it can be positioned at a specific location (cytoplasm, nucleus, etc.) in the cell to measure pH values. In addition, since the pH-responsive fluorescent material on the surface of the nanoprobe 1 is in instantaneous chemical equilibrium with the proton, it is possible to accurately measure in real time the change in a pH value according to the change of time or environment in the cell.

In the present invention, the light source is a laser or LED, etc., and the light incident through the optical fiber 2a may be in the near-infrared or visible region, and may have a wavelength of 300 nm to 1000 nm, and more preferably a wavelength of 400 nm to 700 nm. However, the usable wavelength of light is not limited thereto, and may be arbitrarily selected according to the component, shape and optical properties of the nanoprobe (optical nanowire waveguide), the type of target molecule to be detected, the type of target cell, etc.

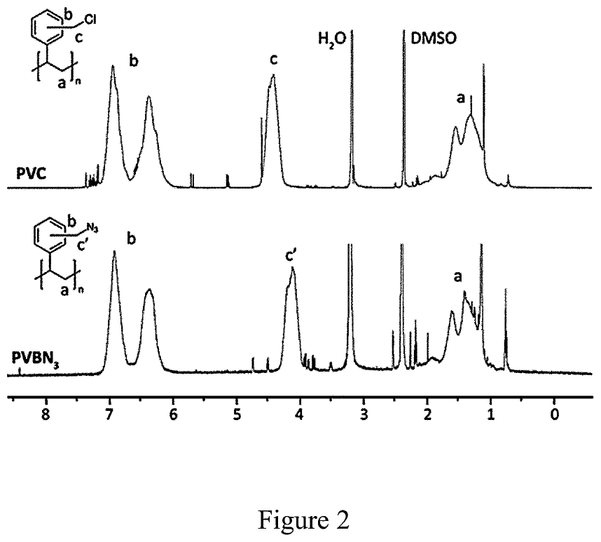

The inventors successfully produced a nanoprobe suitable for in situ monitoring of local pH over time in a single living cell by chemically labelling pH-responsive fluorescent dyes (see Alvarez-Pez, J. M., Ballesteros, L., Talavera, E. & Yguerabide, J. Fluorescein excited-state proton exchange reactions: Nanosecond emission kinetics and correlation with steady-state fluorescence intensity. J. Phys. Chem. A 105, 6320-6332 (2001)) on one end of a polymeric nanowire (FIG. 1a). Here, the pH-responsive fluorescent dyes are a fluorescein molecule having a functional group capable of being conjugated to the nanowire, wherein the fluorescein may be selected from the group consisting of DBCO-FAM, Azide-FAM, Amine-FAM, etc. In detail, by evaporation of PVBN₃ solution (M. 52,000 g/mol) (FIG. 2), an elongated PVBN₃ nanowire was directly grown on the tip of a tapered optical fiber (FIG. 1b and FIG. 3), which was connected to a laser source and a spectrometer by a 1×2 optical fiber coupler (see Methods). As the surface of the nanowire was full of azide moieties (—N₃), its restricted exposure to dibenzocyclooctyne (DBCO)-functionalized fluorescein allowed the terminus of the nanowire (˜500 nm in length) to be selectively modified with high-density pH reporters via a copper-free click reaction (FIG. 4). Importantly, the nanoprobe of the present invention served as a great bidirectional transmission path of the excitation laser (white arrow) and the PL signal (black arrow) from the localized fluorescein. As the PL signal was directly transmitted to the spectrometer, the intensity of the PL spectrum, which changed with proton concentration in a desired location, was measured in real time, regardless of the surroundings of the nanoprobe on the optical fiber (see FIG. 1).

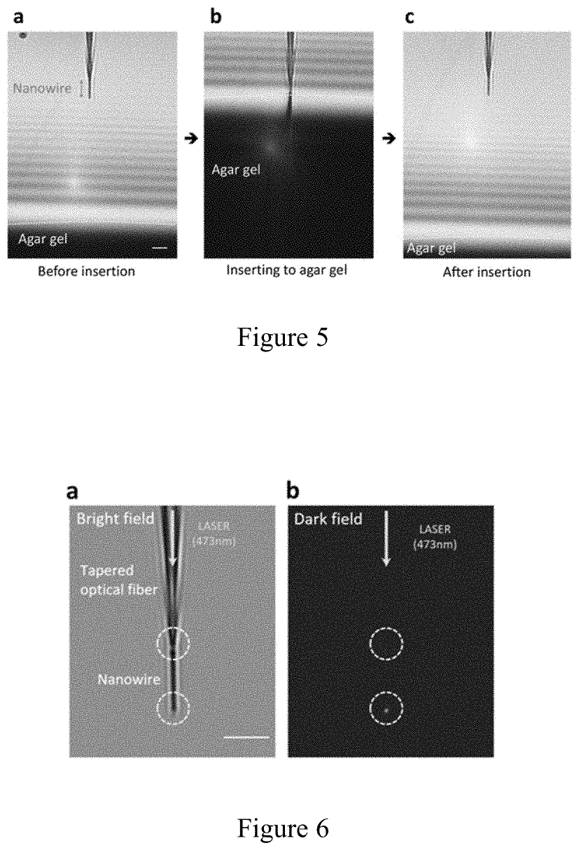

The physical and optical characteristics of the nanoprobe of the present invention were highly compatible for detecting and transmitting the subcellular pH inside a living cell. Based on a previous study of nanowire dimensions minimizing cell damage (see Obataya, I., Nakamura, C., Han, S. W., Nakamura, N. & Miyake, J. Direct insertion of proteins into a living cell using an atomic force microscope with a nanoneedle. Nanobiotechnology 1, 347-352 (2005)), the inventors prepared a nanoprobe with a diameter of ˜200 nm (FIG. 1b), as precisely controlled by a confined-growth method of the present invention (see Je, J. H.; Yang, U.; Oh, S. S.; Yong, M. J.; Kang, B. H. Method of forming micro- or nanowires at predetermined positions of an object using a micro- or nanopipette, U.S. Pat. No. 17,306,220, May 3, 2021). Despite the small diameter, the high Young's modulus of PVBN₃ (E ˜1.7 GPa) (see Bicerano, J. Prediction of Polymer Properties, 2nd ed. (Marcel Dekker, New York, 1996)) allowed the nanoprobe to readily penetrate a rigid matrix; when it was inserted into an agar gel that is known to exhibit a higher Young's modulus (>0.1 GPa) than the actual cellular membrane (E 0.05 GPa) (see Wang, K. et al. Specific membrane capacitance, cytoplasm conductivity and instantaneous Young's modulus of single tumour cells. Sci. Data 4, 1-8 (2017)), no structural deformation was observed (FIG. 5). Importantly, the innate properties of the nanoprobe, including the higher refractive index (˜1.67) (see kJames, J., Hanna, J. M. & Subila, K. B. Refractive Index Engineering using Polymer Nanocomposites. (PhD thesis, University of South Brittany, France, 2019)) of PVBN₃ than that of cellular environments (˜1.37) (see Liu, P. Y. et al. Cell refractive index for cell biology and disease diagnosis: Past, present and future. Lab Chip 16, 634-644 (2016)) and the smooth junction between the nanoprobe and the tapered optical fiber, were suitable for in situ and real-time pH monitoring. Due to negligible scattering in the junction (FIG. 6), the local fluorescent signal of fluorescein was readily collected and transmitted with a coupling efficiency >84% (see Lee, J. et al. Quantitative Probing of Cu²⁺ Ions Naturally Present in Single Living Cells. Adv. Mater. 28, 4071-4076 (2016)).

FIG. 2 shows the ¹H-NMR spectra of PVC (Poly(vinylbenzyl chloride)) and PVBN₃ (Poly(vinylbenzyl azide)).

Upper panel: ¹H spectrum of PVC in DMSO-d₆ that includes aromatic ring (b) at 7.30-6.00 ppm, —CH₂Cl (c) at 4.81-4.38 ppm, and methylene of PVC backbone (a) at 1.87-1.04 ppm. Lower panel: ¹H spectrum of PVBN₃ in DMSO-d₆ that includes aromatic ring (b) at 7.30-6.00 ppm, —CH₂N₃ (c′) at 4.35-3.80 ppm, and methylene (a) at 1.87-1.04 ppm. The shift of —CH₂ from 4.5 ppm (PVC, c) to 4.2 ppm (PVBN₃, c′) shows substitution of chloride by azide, indicating the successful synthesis of PVBN₃. All ¹H-NMR spectra were recorded at 500 MHz using DMSO-d₆ as a solvent at room temperature. The chemical shifts of all H-NMR spectra are referenced to the residual signal of DMSO-d6 (δ 2.50) by BRUKER AVANCE Ascend 500.

FIGS. 3a to 3f show a fabrication procedure of a PVBN₃ nanowire on the tip of a tapered optical fiber. FIG. 3a schematically shows a process of growing PVBN₃ nanowire on the tip of a tapered optical fiber using the apparatus manufactured by the inventors for nanowire fabrication.

FIG. 3b shows an enlarged view of a PVBN₃ nanowire grown on the tip of a tapered optical fiber. The growth process of the PVBN₃ nanowire will be described in detail with reference to FIGS. 3c to 3f First, according to FIG. 3c, a nanowire material solution, that is, a PVBN₃ solution, is filled in a glass nanopipette, and the nanopipette is vertically pulled down to contact the tip of the tapered optical fiber. In the present embodiment, the nanowire material solution is preferably a hydrophobic polymer solution, which may be selected from the group consisting of PVBN₃, PVB-alkyne, and PVB-COOH, etc.

According to FIG. 3d, the tip of the nanopipette is in contact with the tip of the tapered optical fiber. According to FIG. 3e, when the nanopipette is vertically pulled up, a PVBN₃ nanowire grown on the tip of the tapered optical fiber is formed as a solvent of PVBN₃ solution evaporates. FIG. 3f shows a freestanding PVBN₃ nanowire grown on the tip of tapered optical fiber (Scale bar, 10 μm).

FIGS. 4a to 4g show the conjugation process of DBCO-functionalized fluorescein (FAM) to a PVBN₃ nanowire. FIG. 4a schematically illustrates a method of conjugating DBCO-FAM molecule-containing aqueous solution (100 nM), which is a pH-responsive fluorescence dye, with the surface of PVBN₃ nanowire grown on the tip of the tapered optical fiber according to FIGS. 3a to 3f described above. More specifically, according to FIG. 4b, a glass micropipette is filled with DBCO-FAM molecule-containing aqueous solution (100 nM), and then the glass micropipette is vertically pulled down toward the PVBN₃ nanowire such that the tip of the PVBN₃ nanowire is soaked in the aqueous solution in the glass micropipette as shown in FIG. 4c. In FIG. 4c, the DBCO-FAM molecules are conjugated to the azide group of the PVBN₃ nanowire by a copper-free click reaction (see Campbell-Verduyn, L. S. et al. Strain-promoted copper-free ‘click’ chemistry for 18F radiolabeling of bombesin. Angew. Chemie—Int. Ed. 50, 11117-11120 (2011)). In this state, when the micropipette is vertically pulled up, a FAM-labeled nanoprobe is formed as shown in FIG. 4d (Scale bar, 10 μm). FIGS. 4e, 4f and 4g show a bright-field image, a dark-field image, and a merged image of a nanoprobe grown on a tapered optical fiber, obtained by confocal microscopy, respectively (Scale bar, 10 μm). In the dark-field image (FIG. 4f), a green fluorescence signal (shown in white) is clearly observed from the tip of the nanoprobe, but no fluorescence signal is detected in the remaining part of the nanowire and the surface of the tapered optical fiber. The terminal fluorescence is controlled to have a length of 100 nm to 900 nm, preferably 500 nm or less, by precisely adjusting the wetting depth of the nanowire using a high precision x-y-z motor stage with a position accuracy of 250 nm.

FIGS. 5a to 5c show a mechanical property evaluation of the nanoprobe by inserting it into the agar gel. According to FIGS. 5a to 5c, a bright field images before (a), during (b), and after (c) the insertion process are shown, respectively. Here, a white dotted line represents the surface of the agar gel. This analysis confirms that the nanoprobe shows almost no deformation after insertion (Scale bar, 10 μm).

FIGS. 6a to 6b show the optical loss evaluation at the junction between a nanowire and a tapered optical fiber. According to FIGS. 6a to 6b, a bright field image (a) and a dark field image (b) of the nanowire-guided laser light (473 nm) are shown, respectively. Light scattering is observed at the tip of the nanoprobe (lower dashed circles), whereas light scattering is hardly observed at the junction site (white upper dashed circles) (Scale bar, 10 μm).

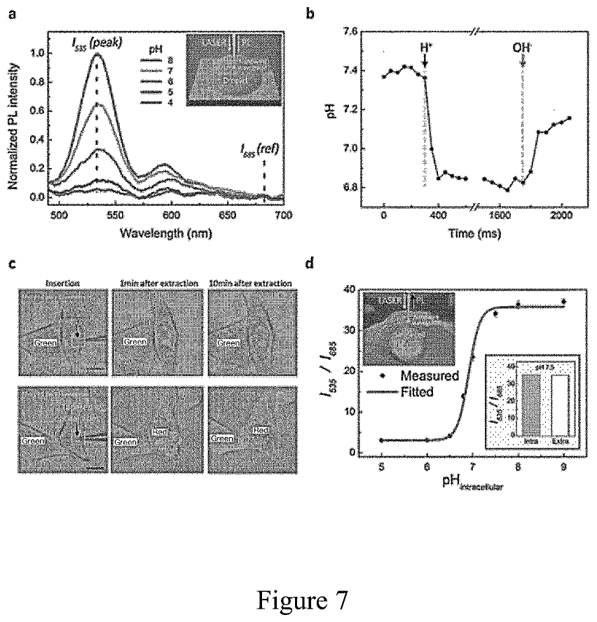

FIGS. 7a to 7d show the optical response of the nanoprobe to the local pH variations. It shows the characterization of the nanoprobe that enables a rapid measurement of pH variations, has a negligible cell damage upon insertion, and selectively responds to pH even in a complex intracellular environment.

According to FIG. 7a, when varying pH from 4 to 8 for a droplet (see inset), the dipped nanoprobe successfully reported pH-dependent PL spectra upon laser excitation (λ\, =473 nm). According to FIG. 7b, time-dependent fluorescent signals (λ=535 nm) were monitored in real time by the nanoprobe due to the quick response to pH variations (<100 ms). Black and gray arrows indicate injection points of acidic and basic solutions, respectively. According to FIG. 7c, the nanoprobe (dotted arrow; diameter ˜200 nm) could be readily inserted into living cells (top), whereas a tapered optical fiber (solid arrow; tip diameter ˜200 nm) caused severe cell damage and leakage (bottom). For the live or dead cell viability assay, the HeLa cells were stained with calcein-AM (green) and propidium iodide (red) (Scale bar, 10 μm). According to FIG. 7d, a pH calibration curve (black) was obtained by measuring the normalized PL peak intensities (I₅₃₅/I₆₈₅) in nigericin-treated cells in pH range of 5 to 9 (n=3), which was followed by fitting with a Boltzmann function (R²=0.9969). As measured in the HeLa cells treated with nigericin at pH 7.5 (n=3), the normalized PL peak intensities at the inside (gray) and outside (white) of the cells were equalized, indicating that intracellular and extracellular pH values were the same (see inset).

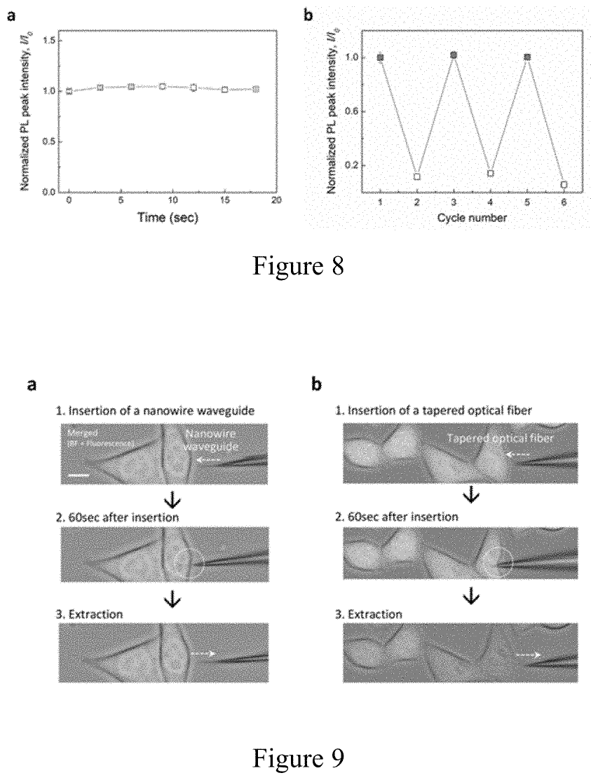

Using the micro-photoluminescence system (FIG. 1a), the inventors investigated pH response of the nanoprobe in solutions with varying pH values from 4 to 8 (FIG. 7a). When the nanoprobe was dipped in different pH droplets (buffer solutions; 5 μl), pH-dependent PL spectra were successfully obtained upon laser excitation (λ=473 nm); the PL peak intensities at 535 nm (I₅₃₅) showed a gradual increment with increasing pH, consistent with the well-known pH-dependent characteristic of fluorescein (see Alvarez-Pez, J. M., Ballesteros, L., Talavera, E. & Yguerabide, J. Fluorescein excited-state proton exchange reactions: Nanosecond emission kinetics and correlation with steady-state fluorescence intensity. J. Phys. Chem. A 105, 6320-6332 (2001)). As the PL peak intensities at 685 nm (I₆₈₅) exhibited negligible variations with increasing pH, they served as reference signals for the remainder of pH monitoring. Importantly, the nanoprobe exhibited excellent photostability and reproducibility in fluorescent detection; against 18 seconds of continuous laser exposure, negligible variation of the PL peak intensity was observed (see FIG. 8), and during cyclic variations in pH between 5.0 and 7.5, the PL peak intensities were reversibly changed (see FIG. 8b).

The PL spectra through the nanoprobe responded to pH variations within a very short time (<100 ms) (FIG. 7b). For instance, when the initially nanowire-dipped droplet with a pH of 7.5 was rapidly changed (acidified) to pH 6.8 (see FIG. 7b, black arrow), the PL peak intensity sharply decreased for times less than 100 ms. Conversely, when this slightly acidic droplet was quickly mixed with a basic buffer solution, the PL peak intensity sharply increased, thereby indicating that the final pH was 7.2 (FIG. 7b, gray arrow). It is well known that as fluorescein reacts instantaneously with a proton, H⁺ (see Alvarez-Pez, J. M., Ballesteros, L., Talavera, E. & Yguerabide, J. Fluorescein excited-state proton exchange reactions: Nanosecond emission kinetics and correlation with steady-state fluorescence intensity. J. Phys. Chem. A 105, 6320-6332 (2001)), the rate-determining step of the pH-responsive behaviour would be proton diffusion in the droplet. Considering that the proton diffusion rate in intracellular fluids is not significantly different from that in buffer solution (see Zaniboni, M. et al. Intracellular proton mobility and buffering power in cardiac ventricular myocytes from rat, rabbit, and guinea pig. Am. J. Physiol. —Hear. Circ. Physiol. 285, 1236-1246 (2003)), the quick fluorescent response to pH variations implies that the nanoprobe system of the present invention would be capable of monitoring pH variations in a real-time manner even in various intracellular environments.

Applicability of the Nanoprobe for pH Monitoring Inside Living Cells

When the nanoprobe of the present invention was injected into living cells, its deep injection did not cause the cells to be severely damaged (see FIG. 7c). For real-time observation of cell viability, HeLa cells were stained with calcein-AM and propidium iodide (PI), which emit green and red fluorescence in live and dead cells, respectively. As attributed to the fine diameter (˜200 nm) and uniform structure (FIG. 1b), the nanoprobe of the present invention did not cause any damage to the cells for 10 minutes after insertion and extraction (FIG. 7c, upper panel). Importantly, evidenced by the lack of a red fluorescence signal, the inventors found that the PI dye did not enter the intracellular space during nanowire insertion or even after nanowire extraction, confirming that the cell membrane was well preserved (see FIGS. 7c and 9). Moreover, the cell morphology was obviously unaffected even after extraction, implying that the pH-sensing system of the present invention is free of membrane rupture and deformation.

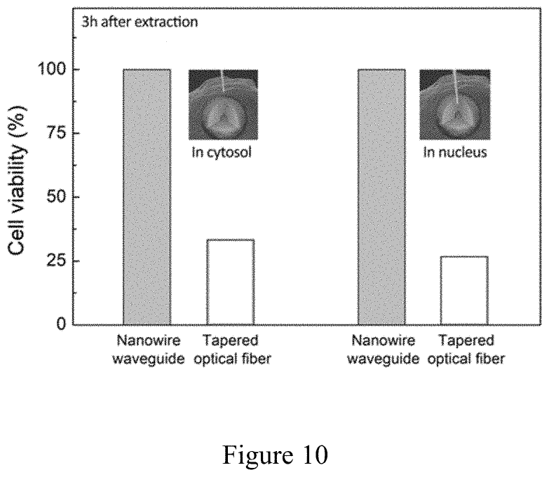

Conversely, the insertion of a tapered optical fiber (tip diameter: ˜200 nm) with a typical conical shape instantly led to cell death due to membrane rupture with leakage of intracellular fluid at the point of insertion (see FIGS. 7c and 9, lower panel). When the inventors compared cell viability by inserting the nanoprobe and the tapered optical fiber into the cytosol and nuclei of HeLa cells, the nanoprobe of the present invention showed definitely higher cell viability (100% in the cytosol (n=20); 100% in the nucleus (n=20)) than the tapered optical fiber (33% in the cytosol (n=20); 25% in the nucleus (n=20)) (see FIG. 10). The inventors found that the cell viability upon insertion of the nanoprobe was much higher than that of existing systems utilized as carriers for bio-sensing probes (see Table 1).

TABLE 1
Comparison of cell viability between the method of the present invention
and previously known methods used to provide various biosensing probes

| Types | Cell viability | Reference documents |
|---|---|---|
| Nanoprobe | 100% | The present invention |
| Tapered optical | 33% | |
| fiber tips | | |
| Tapered optical | 42% | Yan, R. et al. Nanowire-based single- |
| fiber tips | | cell endoscopy. Nat. Nanotechnol. 7, |
| | | 191-196 (2011) |
| bPEI (Branched | 66% | Arif, M., Tripathi, S. K., Gupta, K. C. & |
| polyethylenimine) | | Kumar, P. Self-assembled amphiphilic |
| Lipofectamine | 36% | phosphopyridoxyl-polyethylenimine |
| | | polymers exhibit high cell viability and |
| | | gene transfection efficiency in vitro and |
| | | in vivo. J. Mater. Chem. B 1, 4020-4031 |
| | | (2013) |
| Silica nanoparticle | 90% | Wang, L. et al. A novel cell-penetrating |
| | | Janus nanoprobe for ratiometric |
| | | fluorescence detection of pH in living |
| | | cells. Talanta 209, 120436 (2020) |
| PS-co-PNIP AM | 85% | Liu, H. et al. Dual-emission hydrogel |
| hydrogel | | nanoparticles with linear and reversible |
| | | luminescence-response to pH for |
| | | intracellular fluorescent probes. Talanta |
| | | 211, 120755 (2020) |
| GO glycosheets | 65% | Ji, D. K. et al. Targeted Intracellular |
| | | Production of Reactive Oxygen Species |
| | | by a 2D Molybdenum Disulfide |
| | | Glycosheet. Adv. Mater. 28, 9356-9363 |
| | | (2016) |

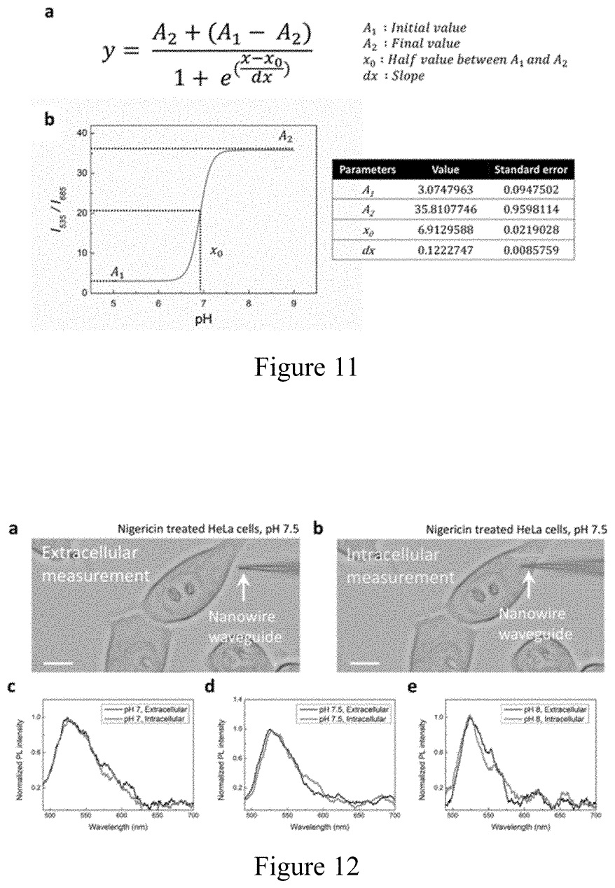

Next, the inventors validated that pH monitoring through the nanoprobe ensures high accuracy even in the presence of complex cellular environment (FIG. 7d). To systematically manipulate the intracellular pH, HeLa cells were incubated in high-potassium buffer solutions with different pH values (pH 5-9), including the K⁺/H⁺-ionophore nigericin (see Llopis, J., McCaffery, J. M., Miyawaki, A., Farquhar, M. G. & Tsien, R. Y. Measurement of cytosolic, mitochondrial, and Golgi pH in single living cells with green fluorescent proteins. Proc. Natl. Acad. Sci. U S. A. 95, 6803-6808 (1998)). By measuring the fluorescence signal intensity ratio (I₅₃₅/I₆₈₅) in nigericin-treated HeLa cells at the fixed pH value, the inventors successfully obtained a pH calibration curve for intracellular pH monitoring (black curve line in FIG. 7d, and FIG. 11). From this curve, the inventors found that the detectable range (pH 6.5˜7.5) of the nanoprobe of the present invention is perfectly suitable for reporting the physiological pH of living cells (see Jaworska, A., Malek, K. & Kudelski, A. Intracellular pH—Advantages and pitfalls of surface-enhanced Raman scattering and fluorescence microscopy—A review. Spectrochim. Acta—Part A Mol. Biomol. Spectrosc. 251, 119410 (2021)). Interestingly, in the nanoprobe of the present invention, almost the same PL intensities were measured at the inside and outside of a living cell under the same pH 7.5 regardless of the surrounding environment (right inset in FIG. 7d, and FIGS. 12 a,b,d); in three experimental groups (pH 7, 7.5, and 8), the PL intensities obtained at the inside and outside of the HeLa cell were also almost the same (FIGS. 12 c,e). This observation suggests that the nanoprobe of the present invention can accurately respond to pH variations even in complex cellular environments containing various ions, proteins, and metabolites (see Ellis, R. J. Macromolecular crowding: An important but neglected aspect of the intracellular environment. Curr. Opin. Struct. Biol. 11, 114-119 (2001)).

FIGS. 8a to 8b show the results of the photostability and reproducibility tests of the nanoprobe. According to FIG. 8a, during continuous laser exposure (473 nm) into a nanoprobe dipped in a buffer droplet (1×PBS, pH 7.4), the time-dependent variation in a PL peak intensity (I/I₀) was negligible, where I₀ is the PL peak intensity at t=0. FIG. 8b shows the results measured in an acid droplet (1×PBS, pH 5.0) and a basic droplet (1×PBS, pH 7.5), alternatively. The inventors found that the reproducibility of the nanoprobe of the present invention was excellent from the cyclic pH variations (n=3) between pH 5.0 (white) and pH 7.5 (black).

FIGS. 9a to 9b show a comparison of cell viability between insertions of the nanoprobe and a tapered optical fiber into living HeLa cells. Here, as indicators of living and dead cells, the cells were stained with calcein-AM (green fluorescence) and propidium iodide (red fluorescence). FIGS. 9a and 9b show Merged (bright field and fluorescence) images during the insertion and the extraction of the nanoprobe (FIG. 9a) or the tapered optical fiber (FIG. 9b). (Scale bar, 10 μm).

FIG. 10 shows a histogram of cell viability after the extraction of the inserted nanoprobe (gray) or the tapered optical fiber (white) from cytosol and nucleus, respectively (n=20). HeLa cells were stained with propidium iodide dye, as the indicator of dead cells. Cell viability is based on the number of live cells with no red fluorescence signal.

FIGS. 11a to 11b show a Boltzmann fitting for obtaining a pH calibration curve targeting intracellular environments. FIG. 11a shows an Equation of Boltzmann fitting for the pH calibration curve. FIG. 11b shows a Boltzmann fitting curve with intensity ratio (I₅₃₅/I₆₈₅) as a function of pH values of nigericin-treated HeLa cells (left graph), and values of each parameter (right table).

FIGS. 12a to 12e show the results of investigation for pH-dependent fluorescent signals of the nanoprobes at the outside and inside of living HeLa cells. FIGS. 12a and 12b show bright field images (fixed pH: 7.5) of nanowire insertion sites at the outside (a) and inside (b) of HeLa cell, respectively. FIGS. 12c to 12e show PL spectra of the nanoprobe measured at the outside (black line) and inside (gray line) of nigericin-treated HeLa cells at varying pH (7.0-8.0) (Scale bar, 10 μm).

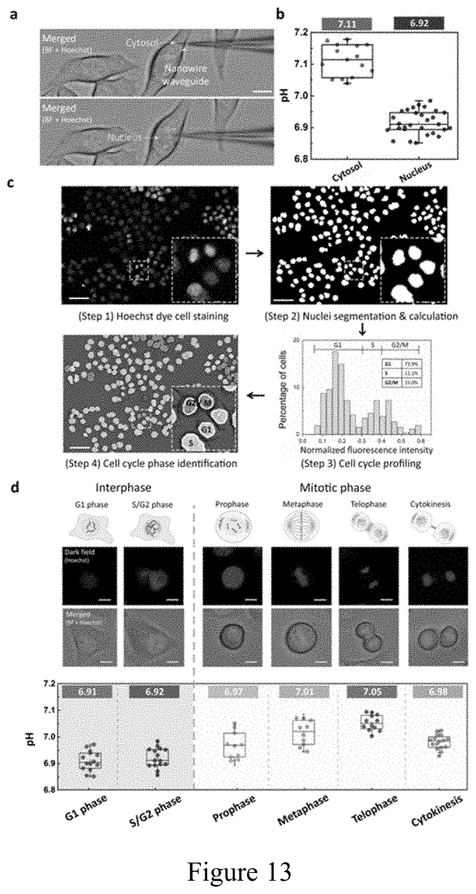

FIGS. 13a to 13d show the results of pH value monitoring in the cytosol and the nuclei during an entire cell cycle of a single cell using a nanoprobe. According to FIG. 13a, the nanoprobe was inserted into the cytosol (top) and nuclei (bottom) of living HeLa cells that were stained with Hoechst dyes (Scale bar, 10 μm). FIG. 13b is a comparison view between cytosolic pH (n=15) and nuclear pH (n=29). FIG. 13c shows a identification of cell cycle stages for individual HeLa cells. When Hoechst dyes specifically stained nuclei of living cells (Step 1), the net fluorescence intensities of the nuclei were calculated for all the cells using the automated image segmentation algorithm of the present invention (Step 2), and a DNA histogram was prepared to profile the cell cycles of HeLa cells (Step 3), and the phase of each cell was identified by colour mapping on the cell image (Step 4) (Scale bar, 50 μm). FIG. 13d shows nuclear pHs measured for each cell cycle phase. As schematics of cell cycle phases (top), dark field and merged (bright field+fluorescence) images of Hoechst-stained cells (middle), and nuclear pH values (bottom) are displayed for each cell cycle phases. G1 and S/G2 phases showed similar pH values (G1 phase: 6.91±0.03 (n=14); S/G2 phase: 6.92±0.03 (n=15)), while the nuclear pH values in prophase, metaphase, telophase, and cytokinesis exhibited a tidal curve (prophase: 6.97±0.05 (n=10); metaphase: 7.01±0.05 (n=10); telophase: 7.05±0.03 (n=12); cytokinesis: 6.98±0.03 (n=16)) (Scale bar, 10 μm).

As the nanoprobe of the present invention is able to monitor the local pH of different organelles in real time, the inventors were able to successfully demonstrated the measurement of pH values for the cytosol and nuclei within single living cells (FIGS. 13a to 13b). Despite the importance of the nuclear pH in regulating critical cellular functions (e.g., DNA replication, gene expression, and epigenetic modulation) (see Francastel, C., Schübeler, D., Martin, D. I. K. & Groudine, M. Nuclear compartmentalization and gene activity. Nat. Rev. Mol. Cell Biol. 1, 137-143 (2000); and Nakamura, A. & Tsukiji, S. Ratiometric fluorescence imaging of nuclear pH in living cells using Hoechst-tagged fluorescein. Bioorganic Med. Chem. Lett. 27, 3127-3130 (2017)), it was extremely difficult to measure (determine) directly the nuclear pH (see Casey, J. R., Grinstein, S. & Orlowski, J. Sensors and regulators of intracellular pH. Nat. Rev. Mol. Cell Biol. 11, 50-61 (2010)), which is mostly because of the presence of two robust compartmentalizing membranes: the cellular membrane and the nuclear envelope. Due to the large-diameter (˜120 nm) nuclear pores within the nuclear envelope, a number of studies have assumed that pH in the nucleus is identical to that in the cytosol (see Casey, J. R., Grinstein, S. & Orlowski, J. Sensors and regulators of intracellular pH. Nat. Rev. Mol. Cell Biol. 11, 50-61 (2010); and Fabre, E. & Hurt, E. C. Nuclear transport. Current Opinion in Cell Biology vol. 6 (EMBL, Heidelberg, 1994)). However, based on recent efforts over the past decade, it was suggested that the nuclear compartment can control its own internal pH, thereby making the nuclear pH differ from the cytosolic pH (see Sherman, T. A., Rongali, S. C., Matthews, T. A., Pfeiffer, J. & Nehrke, K Identification of a nuclear carbonic anhydrase in Caenorhabditis elegans. Biochim. Biophys. Acta—Mol. Cell Res. 1823, 808-817 (2012); Santos, J. M., Mart Inez-Zaguilan, R., Facanha, A. R., Hussain, F. & Sennoune, S. R. Vacuolar H+-ATPase in the nuclear membranes regulates nucleo-cytosolic proton gradients. Am. J. Physiol. —Cell Physiol. 311, C547-0558 (2016); and Nakamura, A. & Tsukiji, S. Ratiometric fluorescence imaging of nuclear pH in living cells using Hoechst-tagged fluorescein. Bioorganic Med. Chem. Lett. 27, 3127-3130 (2017)). To answer this controversial question, the inventors separately measured pH values of the nucleus and the cytosol by inserting the nanoprobe into the desired sites of single living HeLa cells (FIG. 13a). As a result, the inventors found that the nuclear pH (6.92±0.04, n=29) was meaningfully lower than the cytosolic pH (7.11±0.05, n=15) (FIG. 13b), implying that there could be a pH gradient between the nucleus and the cytosol by separate pH regulatory functions for each cell compartment.

As the robust membrane of the nucleus was easily penetrated by the nanoprobe without leakage, the inventors were able to directly monitor nuclear pH variations throughout the entire human cell cycle. For this, preliminarily, it was necessary to identify the cell cycle status of individual HeLa cells (FIGS. 13c-13d) (see Rloukos, V., Pegoraro, G., Voss, T. C. & Misteli, T. Cell cycle staging of individual cells by fluorescence microscopy. Nat. Protoc. 10, 334-348 (2015)); in principle, as cell division progresses, the total DNA quantity inside the nucleus varies, where quantification of the total DNA quantity can determine the cell cycle stage of cell division. In detail, the inventors first stained the cells with Hoechst dye, which emits a blue fluorescent signal by specifically conjugating to DNAs inside the nuclei (Step 1), and the DNA content of each cell was then measured by automated image analysis (nuclei segmentation), in which the total fluorescence intensities were calculated for a number of nuclei (FIG. 13c, see Methods). Finally, the cell cycle stage was identified by colour mapping on the cell image based on the DNA histogram (Step 4). From the analysis, the inventors ascertained the cell cycle phase (G1, S, and G2/M) of individual HeLa cells and subsequently obtained the ratio of each phase (G1, 73.9%; S, 11.1%; G2/M, 15.0%), which was well matched to the reported characteristics of HeLa cells (G1, 72.1%; S, 12.6%; G2/M, 12%) (see Athukorala, Y., Trang, S., Kwok, C. & Yuan, Y. V. Antiproliferative and antioxidant activities and mycosporine-Like amino acid profiles of wild-Harvested and cultivated edible canadian marine red macroalgae. Molecules 21, (2016)).

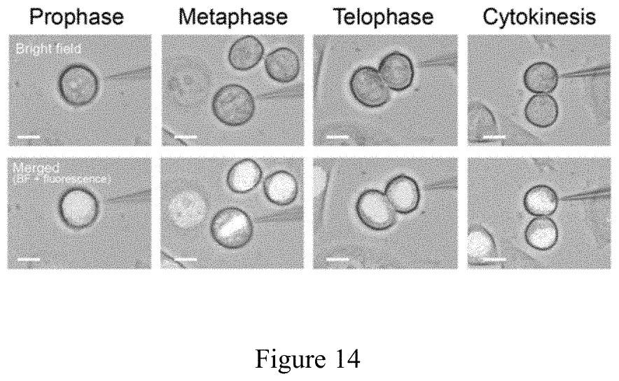

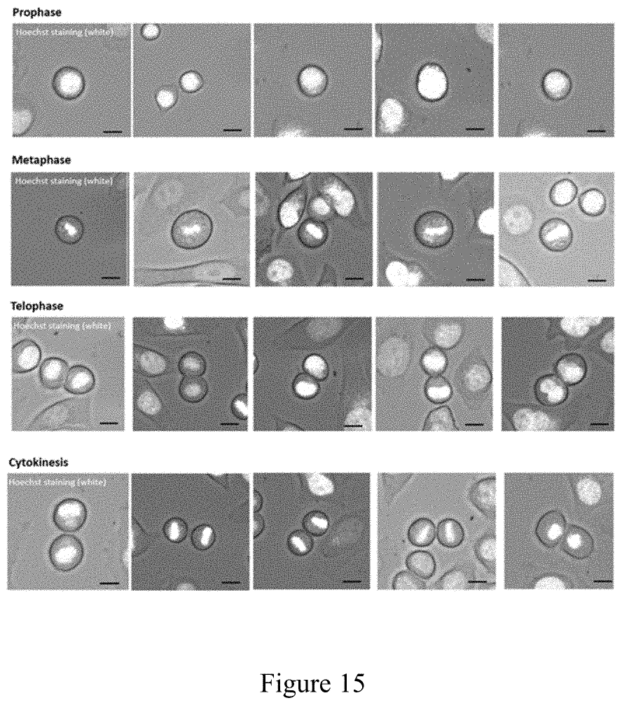

Based on the assessment of each cell cycle stage (FIG. 13c), the inventors then measured nuclear pH variations during cell division, discovering pH homeostasis in interphase and pH fluctuation in the mitotic phase (FIG. 13d). Specifically, the HeLa cells in the G1 and S/G2 phases exhibited similar pH values (G1 phase: 6.91±0.03 (n=14); S/G2 phase: 6.92±0.03 (n=15), FIG. 13d, gray box). Previously, it was reported by several studies that during interphase, the cytosol displayed pH fluctuations for several reasons, such as ATP synthesis/hydrolysis and redox oscillations (see Da Veiga Moreira, J. et al. Cell cycle progression is regulated by intertwined redox oscillators. Theor. Biol. Med. Model. 12, 1-14 (2015); and DeBerardinis, R. J., Lum, J. J., Hatzivassiliou, G. & Thompson, C. B. The Biology of Cancer: Metabolic Reprogramming Fuels Cell Growth and Proliferation. Cell Metab. 7, 11-20 (2008)). Unlike before, the inventors clearly observed that the nucleus preserved its own pH without pH variation in G1 and S/G2 phases, presumably due to the pH-regulating function of the nuclear membrane; this nuclear pH homeostasis in the interphase is consistent with the previous finding that investigated the nuclear pH changes of budding yeast (see Zhao, H. et al. Dynamic imaging of cellular pH and redox homeostasis with a genetically encoded dual-functional biosensor, pHaROS, in yeast. J. Biol. Chem. 294, 15768-15780 (2019)). Strikingly, when the HeLa cells entered prophase, the nuclear pH continued to slightly increase until the cells reached telophase (prophase: 6.97±0.05 (n=10); metaphase: 7.01±0.05 (n=10); telophase: 7.05±0.03 (n=12), white box at the bottom in FIG. 13d, and FIGS. 14-15). During the mitotic phase, the transient disruption of nuclear pH homeostasis might be related to the breakdown of the nuclear envelope (see Cooper G M. The Cell: A Molecular Approach. 2nd edition. (Sunderland (Mass.), Sinauer Associates, 2000)), temporarily interrupting pH regulation abilities of the nucleus. However, as the cells arrived at a cytokinesis phase at the end of mitosis, the nuclear pH returned to its original pH value (cytokinesis: 6.98±0.03 (n=16)), suggesting that the reconstruction of divided cell nucleus envelopes would lead to the recovery of original pH homeostasis. By direct pH monitoring during the entire cell cycle, it was evident that the nucleus serves its own pH-regulating function.

FIG. 14 is photographs of bright field images (upper panel) and merged (bright field and fluorescence, lower panel) images for the insertion of a nanoprobe into single living HeLa cells during mitotic phase, observed by confocal microscopy (Scale bar, 10 μm).

FIG. 15 shows merged (bright field and fluorescence) images of HeLa cells, stained with nucleus-specific Hoechst dye (white), during mitotic phase (from prophase to cytokinesis) for pH measurement, observed by confocal microscopy (Scale bar, 10 μm).

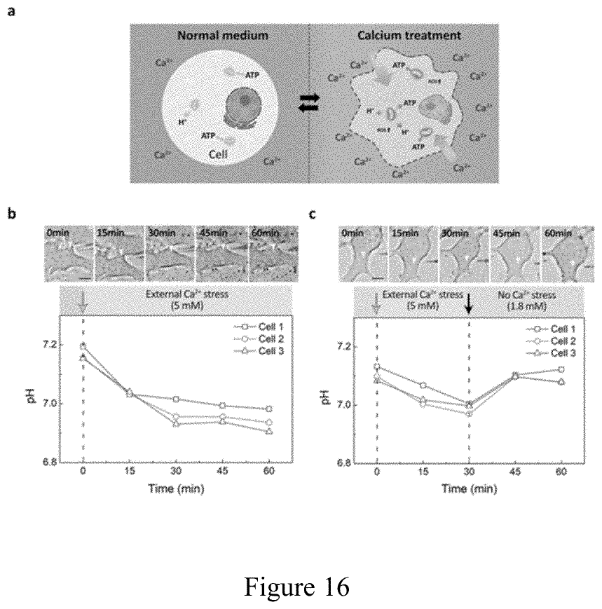

FIGS. 16a to 16c show cytosolic pH variations in response to external calcium ions, which shows the results of real-time cytosolic pH monitoring for ion stresses. FIG. 16a shows a schematic illustration of intracellular acidification in the presence of excessive calcium ions. In general, high concentrations of calcium ions elicit adverse effects on cells, including overproduction of adenosine triphosphates (ATPs) and reactive oxygen species (ROS), thereby affecting pH homeostasis. According to FIGS. 16b to 16c, different response of individual cells against external calcium ion stress were measured by cytosolic pH variations (n=3). White triangles in brightfield images indicate positions where the nanoprobe of the present invention was inserted for pH measurements. Gray and black arrows indicate introduction and removal points of external Ca²⁺ stress, respectively (Scale bar, 10 μm).

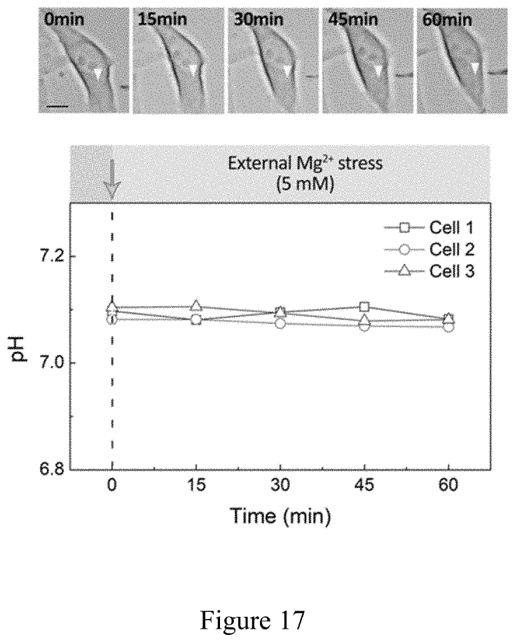

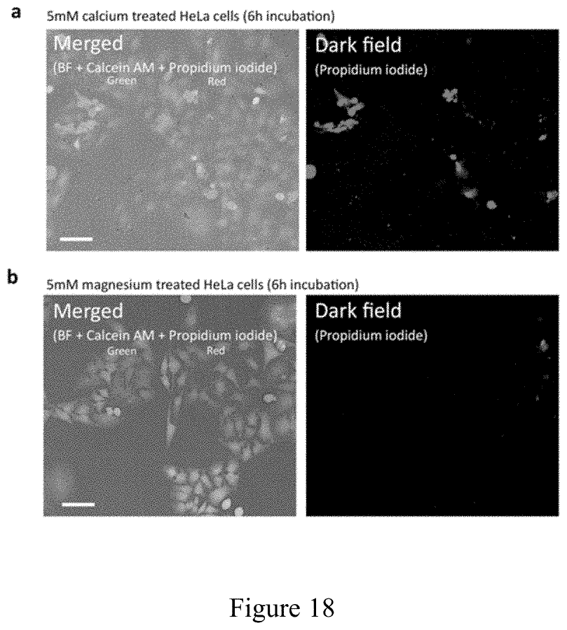

Additionally, as a result of investigating the cytosolic pH dynamics of single living HeLa cells by providing external divalent ion stresses, the inventors confirmed that individual cells actually react differently depending on the ion (FIGS. 16a to 16c). When excessive amounts of calcium (5 mM) were added to the cell culture medium, the cytosolic pH decreased significantly (7.17±0.02 to 6.97±0.04) within half an hour as a result of intracellular acidification induced by high extracellular Ca²⁺ (FIG. 16b). Interestingly, when Ca²⁺ was substituted with Mg²⁺, there were negligible pH variations (7.09±0.01 to 7.08±0.01)(FIG. 17). It is known that the presence of excess Ca²⁺ in extracellular medium can elicit the generation of reactive oxygen species (ROS), mitochondrial dysfunction by increasing ATP levels, and even cell death through apoptosis and necrosis (see McGinnis, K. M., Wang, K. K. W. & Gnegy, M. E. Alterations of extracellular calcium elicit selective modes of cell death and protease activation in SH-SY5Y human neuroblastoma cells. J. Neurochem. 72, 1853-1863 (1999); and Voccoli, V., Tonazzini, I., Signore, G., Caleo, M. & Cecchini, M. Role of extracellular calcium and mitochondrial oxygen species in psychosine-induced oligodendrocyte cell death. Cell Death Dis. 5, 1-10 (2014)). Accordingly, it was considered that the Ca²⁺-dependent intracellular acidification of HeLa cells occurred by the adverse effects of the high extracellular Ca²⁺, which was further supported by scrutinizing cell viabilities depending on calcium ion treatments (FIG. 18a). Moreover, the magnesium treatment experiment of the present invention revealed that the cells were tolerant to increases in extracellular Mg²⁺ concentrations, and consistent with a previous report (see Libako, P. et al. Blocking the rise of intracellular calcium inhibits the growth of cells cultured in different concentrations of magnesium. Magnes. Res. 25, 12-20 (2012)), there were no cellular malfunctions or cell deaths, unlike with Ca²⁺ stimulation (FIGS. 17 and 18b).

Importantly, living HeLa cells restored their original pH state when the external ion stress was removed (FIG. 16c). To observe the recovery in pH homeostasis, the inventors incubated HeLa cells with excess amounts of Ca²⁺ (5 mM) for 30 min and then quickly adjusted the Ca²⁺ concentration of the medium to the normal range (1.8 mM). During this process, the inventors monitored cytosolic pH changes in three individual cells. As observed from the previous Ca²⁺-dependent intracellular acidification (FIG. 16b), for the first 30 min, high extracellular Ca²⁺ induced the cytosol of HeLa cells to be acidic (7.10±0.02 to 6.99±0.02). Surprisingly, after the removal of extracellular Ca²⁺ stress (FIG. 16c, black arrow), the cells gradually restored their intrinsic neutral pH (6.99±0.02 to 7.09±0.02), meaning that the cytosolic pH homeostasis of living HeLa cells was successfully recovered from the loss of pH control, which was previously caused by ionic stress. It was interesting that the overall tendencies of HeLa cells against external ion stresses were similar, but individual of responses HeLa cells expressed as pH were heterogeneous, such as cell-to-cell differences such as size, morphology, neighbouring cells, and dividing phases (see Kültz, D. Molecular and evolutionary basis of the cellular stress response. Annu. Rev. Physiol. 67, 225-257(2005)).

FIG. 17 shows the result of real-time measurements of cytosolic pH of HeLa cells treated by excessive magnesium ion (5 mM), and shows bright field images (upper panel) and measured pH variations (lower graph), obtained by tracking a single HeLa cell (n=3). White triangles in the upper panel indicate the insertion position of the nanoprobe. Gray arrow indicates the point of exchanging the medium from a normal magnesium concentration (1.8 mM) to the high magnesium concentration (5 mM) magnesium concentrations (Scale bar, 10 μm).

FIGS. 18a to 18b show merged (bright field and fluorescence) images and dark field images of living HeLa cells treated by excessive calcium ion (a) and excessive magnesium ion (b), respectively. In both assays, HeLa cells were pre-stained with calcein-AM and propidium iodide to analyze cell viability (Scale bar, 100 μm). Accordingly, the inventors could found that the calcium ion treatment puts stress on the HeLa cells and lowers the cell viability, but the magnesium ion treatment did not affect the cell viability of HeLa cells.

According to the present invention, by utilizing the nanoprobe with local pH-detecting and transmitting function, the inventors were able to successfully access organelle and cytosol to monitor their pH dynamics in single living cell without causing cell damage and leakage. Beyond impermeable cellular and nuclear membranes, the in situ pH monitoring of the present invention is significant in that it can provide a fundamental understanding of the role of subcellular organelle membranes. From the observation of pH difference between the cytosol (7.11±0.05) and the nucleus (6.92±0.04), it has been confirmed that cellular activities can exhibit different pH dynamics by nuclear membranes (see Sherman, T. A., Rongali, S. C., Matthews, T. A., Pfeiffer, J. & Nehrke, K Identification of a nuclear carbonic anhydrase in Caenorhabditis elegans. Biochim. Biophys. Acta—Mol. Cell Res. 1823, 808-817 (2012); Santos, J. M., Martinez-Zaguilan, R., Facanha, A. R., Hussain, F. & Sennoune, S. R. Vacuolar H+-ATPase in the nuclear membranes regulates nucleo-cytosolic proton gradients. Am. J. Physiol. —Cell Physiol. 311, C547-0558 (2016); and Nakamura, A. & Tsukiji, S. Ratiometric fluorescence imaging of nuclear pH in living cells using Hoechst-tagged fluorescein. Bioorganic Med. Chem. Lett. 27, 3127-3130 (2017)). In particular, pH homeostasis and fluctuation for cellular growth and division in the nucleus infer that before breakdown, the nuclear envelope is involved in pH maintenance, as well as nuclear transport, in facilitating biosynthetic activities of the cell (see Cooper G M. The Cell: A Molecular Approach. 2nd edition. (Sunderland (Mass.), Sinauer Associates, 2000); and Demaurex, N. pH homeostasis of cellular organelles. News Physiol. Sci. 17, 1-5 (2002)). To the best of the inventors' knowledge, this is the first direct evidence for the existence of an independent pH-control function, especially in the dividing nucleus of human cells.

As observed by different cellular responses to external ionic stimuli, the local pH-monitoring nanoprobe of the present invention would be widely applicable for studying an individual cell's life under diverse interesting conditions. For instance, real-time detection of pH-variations in organelles during various cellular behaviours (e.g., differentiation, cell signalling or communication, and programmed cell death) could be used to understand biological processes along organelle membranes (see Jaworska, A., Malek, K. & Kudelski, A. Intracellular pH—Advantages and pitfalls of surface-enhanced Raman scattering and fluorescence microscopy—A review. Spectrochim. Acta—Part A Mol. Biomol. Spectrosc. 251, 119410 (2021); and Han, J. & Burgess, K. Fluorescent indicators for intracellular pH. Chem. Rev. 110, 2709-2728 (2010)).

Methods

Reagents and Materials.

Poly(vinylbenzyl chloride) (PVC, Mₙ=55,000 g/mol), sodium azide, N,N-dimethylformamide (DMF), 1-methyl-2-pyrrolidinone (NMP), methanol, dimethyl sulfoxide-d₆ (DMSO-d₆), agar powder, 10× phosphate-buffered saline (PBS), sodium hydroxide, hydrochloric acid (37%), propidium iodide, calcein-AM, and nigericin sodium salt were purchased from Sigma-Aldrich (St. Louis, Mo.). 5′-DBCO-T₅-FAM-3′ was synthesized by Bioneer (Daejeon, Korea). HEPES (pH 7.5) buffer (1 M), potassium chloride, calcium dichloride (CaCl₂)) and magnesium dichloride (MgCl₂) were purchased from BioPrince (Chuncheon, Korea). Hoechst 33342 (10 mg/ml) solution was purchased from Biotium (Fremont, Calif.). Glass capillaries (BF-100-50-10) for nanopipette fabrication were purchased from Sutter Instrument (Novato, Calif.).

Fabrication of Nanoprobes.

A mixture of PVC (0.014 g, 131 mmol) and sodium azide (0.010 g, 220 mmol) in anhydrous DMF solvent (0.7 mL) was fluxed in an amber vial at 70° C., which was covered by aluminum foil to block light. After 2 hours of reaction, methanol (0.5 mL) was added, and then the mixed solution was centrifuged (Mini microcentrifuge, Labogene) at 10,000 rpm for 1 min to remove excess unreacted reagents and precipitate an azide-functionalized polymer. Finally, the obtained precipitates were dried under vacuum for 1 hour and were then dissolved with the addition of NMP solvent (50 μL). The successful synthesis of PVBN₃ was confirmed by ¹H NMR spectroscopy (FIG. 2). For nanowire fabrication, glass nanopipettes were processed using a P-97 micropipette puller (Sutter Instrument), and tapered optical fibres were manufactured using a P-2000 laser-based micropipette puller (Sutter Instrument). Thereafter, the positions of the glass nanopipette and the tapered optical fibre were precisely controlled by x-y-z stepping motor stages, with a position accuracy of ˜250 nm (Kohzu Precision). For nanowire fabrication, a glass nanopipette, which was filled with a PVBN₃ solution in NMP at a concentration of 1.0 wt %, was pulled down in the vertical direction to touch the tip of a tapered optical fibre. As the nanopipette was pulled up in the vertical direction, a PVBN₃ nanowire was formed on the tip of the tapered optical fibre by rapid solvent evaporation, forming a freestanding PVBN₃ nanowire. The fabrication of the nanowire was monitored in real time using the optical imaging system of the present invention consisting of two-axis CCD cameras (INFINITY 1-2C, Lumenera Camera), an objective lens (100× Plan Apo Infinity Corrected Objective, Mitutoyo), and yellow LED illuminators (Precision LED spotlight, 590 nm, Mightex).

Conjugation of Fluorescein to the Nanowire

For conjugation of DBCO-functionalized fluorescein (FAM) to the PVBN₃ nanoprobe, a glass micropipette was filled with DBCO-FAM molecule-containing aqueous solution (100 nM). When the glass micropipette was pulled down in the vertical direction to soak the nanowire, the DBCO-FAM molecule was conjugated to the azide group of the PVBN₃ nanowire for 10 min by a click reaction. By adjusting the contact area between the nanoprobe and the DBCO-FAM molecule-containing solution, the inventors were able to control the FAM-labelled region of the nanoprobe. Before pH measurement assay, the nanoprobe was washed twice with 1×PBS solution.

Measurement of Fluorescent Signals (PL Spectra) from the Nanoprobe.

To excite the DBCO-functionalized fluorescein at the end of the nanoprobe, a continuous laser (473 nm blue solid-state laser, MBL-III-473, Uniotech), combined with a computer-controlled shutter, was injected into the nanowire through the optical fibre and a 1×2 optic coupler (narrowband fibre optic coupler, 532±15 nm, 50:50 split, Thorlab). All PL spectra were recorded by a spectrometer (Avaspec-ULS2048L-EVO, Avantes).

Cell Culture Experiment.

HeLa cells were obtained from Korean Cell Line Bank. Cells were cultured in Dulbecco's modified Eagle's medium (DMEM, Welgene) supplemented with 10% fetal bovine serum (FBS, Gibco), 100 U/ml penicillin (Welgene), and 100 μg/ml streptomycin (Welgene) in a 35-mm culture dish (SPL Life Sciences) under the proper conditions (37° C. temperature and 5% CO₂ atmosphere). When preparing for cell experiments, the inventors cultured HeLa cells for two days.

Cell Viability Assay.

To analyse cell viability, HeLa cells were preincubated with calcein-AM and propidium iodide dyes at 37° C. for 15 min. To investigate the insertion effect of the nanoprobe and the tapered optical fibre on HeLa cells, both were inserted into the cytosol or nucleus of HeLa cells for 1 min and then extracted. After this process, cell viability was evaluated through the observation of green fluorescence (515 nm) and red fluorescence (636 nm) by confocal microscopy (STELLARIS 5, Leica) with a 10× objective lens (0.4 numerical aperture, HC PL APO 10×, Leica). In the cell viability histogram investigation, cells were incubated under cell culture conditions for 3 h and then imaged by confocal microscopy.

Manipulation of Intracellular pH to Obtain a Calibration Curve.

The cultured HeLa cells were washed twice with freshly prepared DMEM and nigericin buffer (10 mM HEPES, 10 mM NaCl, 130 mM KCl, 1 mM MgCl₂) at varying pH values (5-9). Next, the inventors added 15 μM nigericin to the washed cells at 37° C. for 15-25 min. Based on the sigmoidal increase of the intensity ratio relying on different pH-dependent fluorescent signals (5-9), the pH calibration curve was obtained by Boltzmann fitting with a good correlation to measured data (R²=0.9969), a very good sensitivity (18.722 (I₅₃₅/I₆₈₅)/pH units), and a detecting resolution (0.0365 pH units), calculated by the IUPAC definition.

Setup for Insertion of the Nanoprobe into Single HeLa Cells.

Before cell experiments, the cultured HeLa cells were washed twice with freshly prepared DMEM. To precisely control the insertion site of the nanoprobe inside a single HeLa cell, the inventors accurately positioned the nanoprobe using the microphotoluminescence setup of the present invention, consisting of an x-y-z micromanipulator (positioning accuracy: 250 nm, Kohzu Precision), motor controllers (SC-210, Kohzu Precision) and computers. During insertion, the position of the nanoprobe was monitored by confocal microscopy (STELLARIS 5, Leica) with a 10× objective lens (0.4 numerical aperture, HC PL APO 10×, Leica) and a CCD camera. While the nanoprobe was positioned to a desired site inside the cell, PL spectra were collected in real time.

Identification of the Cell Cycle Status of Individual HeLa Cells.

To specifically stain DNA from HeLa cells, the inventors first prepared diluted Hoechst 33342 solution (10 μg/ml) and then mixed it with cultured cells for 15 min (at the cell culture conditions). Images of stained cells were acquired by confocal microscopy at 2048×2048 pixels. By applying the MATLAB-based image processing algorithm for nuclei segmentation, the nuclear fluorescence intensity of each cell was calculated. In detail, the algorithm was designed to remove noise from raw images using Gaussian filtering and binarize the filtered images by setting an adaptive thresholding. Then, the binarized images were segmented by smoothing rough edges by applying the opening & closing algorithm. To minimize the identification error of individual nucleus segmentation, small binary noise clusters and nuclei around the border regions of the image process were automatically removed. Based on automatically segmented images, the fluorescence intensity within the segmented region of each nucleus was collected. From the fluorescence intensity data, the DNA histogram was plotted in which individual cells were classified in different cell cycle phases (G1, S, G2/M) by visually selected cut-offs (see Roukos, V., Pegoraro, G., Voss, T. C. & Misteli, T. Cell cycle staging of individual cells by fluorescence microscopy. Nat. Protoc. 10, 334-348 (2015)). Here, the percentage of cells within each phase was automatically calculated using Origin software (version 8.5). The phase of each cell in the images was identified as G phase, S phase, and G2/M phases by colour mapping to the cell image with different colours based on the DNA histogram.

Measurement of Nuclear pH Variation During the Cell Cycle.

The cultured HeLa cells were washed twice with freshly prepared DMEM and incubated with Hoechst dye-containing buffer (10 μg/ml in DMEM) for 15 min. After the medium was changed to fresh DMEM buffer, the nuclear pH was measured by insertion of the nanoprobe into single living HeLa cells in each cell cycle phase and imaging by confocal microscopy.

Although various embodiments of the present invention have been described above, the embodiments have been described so far are merely illustrative of some of the preferred embodiments of the present invention, and the scope of the present invention is not limited by the embodiments described above, except for the appended claims. Accordingly, it is understood that those having ordinary knowledge in the same technical field can make many changes, modifications and substitutions of equivalents without departing from the technical spirit and gist of the invention within the scope of the following claims.

### LIST OF REFERENCE NUMERALS

1: Nanoprobe
2: Optical fiber
2a: First optical fiber
2b: Second optical fiber
3: Tapered tip
4: Light source
5: Fiber coupler
6: Manipulator
7: Single living cell
8: Spectrometer

What is claimed is:

1. A method of manufacturing a nanoprobe, comprising:
(a) filling a nanopipette with a nanowire material solution and pulling down the nanopipette to bring the nanowire material solution into contact with the tip of an optical fiber;
(b) pulling up the nanopipette to grow a nanowire on a tip of the optical fiber;
(c) filling a micropipette with an aqueous solution containing a pH-responsive fluorescent material and pulling down the micropipette to immerse a part of the nanowire in the aqueous solution; and
(d) pulling up the micropipette to form a nanoprobe labeled with a pH-responsive fluorescent material.

2. The method of manufacturing a nanoprobe according to claim 1, wherein the nanowire material solution is a hydrophobic polymer solution.

3. The method of manufacturing a nanoprobe according to claim 2, wherein the hydrophobic polymer solution is selected from the group consisting of at least PVBN₃, PVB-alkyne, and PVB-COOH.

4. The method of manufacturing a nanoprobe according to claim 1, wherein the optical fiber has a tapered tip.

5. The method of manufacturing a nanoprobe according to claim 1, wherein the pH-responsive fluorescent material is a fluorescein molecule having a functional group capable of being conjugated to the nanowire.

6. The method of manufacturing a nanoprobe according to claim 5, wherein the fluorescein is selected from the group consisting of at least DBCO-FAM, Azide-FAM, and Amine-FAM.

7. The method of manufacturing a nanoprobe according to claim 1, wherein the wetting (or labeled) length of the nanowire by the pH-responsive fluorescent material is controlled to be 100 nm to 900 nm.

8. The method of manufacturing a nanoprobe according to claim 1, wherein the wetting (or labeled) length of the nanowire by the pH-responsive fluorescent material is controlled to be 100 nm to 500 nm.

9. A nanoprobe for pH measurement comprising:
an optical fiber;
a nanowire formed by growing a nanowire material solution at one end of the optical fiber; and
a pH-responsive fluorescent material labeled on a part of the nanowire.

10. The nanoprobe according to claim 9, wherein the nanowire material solution is a hydrophobic polymer solution.

11. The nanoprobe according to claim 10, wherein the hydrophobic polymer solution is selected from the group consisting of at least PVBN₃, PVB-alkyne, and PVB-COOH.

12. The nanoprobe according to claim 9, wherein the optical fiber has a tapered tip at one end.

13. The nanoprobe according to claim 9, wherein the pH-responsive fluorescent material is a fluorescein molecule having a functional group capable of being conjugated to the nanowire.

14. The nanoprobe according to claim 13, wherein the fluorescein is selected from the group consisting of at least DBCO-FAM, Azide-FAM, and Amine-FAM.

15. The nanoprobe according to claim 9, wherein the wetting (or labeled) length of the nanowire by the pH-responsive fluorescent material is controlled to be 100 nm to 900 nm.

16. The nanoprobe according to claim 9, wherein the wetting (or labeled) length of the nanowire by the pH-responsive fluorescent material is controlled to be 100 nm to 500 nm.

17. The nanoprobe according to claim 9, wherein the nanoprobe has a uniform diameter.

18. The nanoprobe according to claim 9, wherein the nanoprobe has a diameter of 10 nm to 900 nm.

19. The nanoprobe according to claim 9, wherein the nanoprobe has a diameter of 10 nm to 400 nm.

20. The nanoprobe according to claim 9, wherein the nanoprobe has a length of 1 μm to 10 μm.

21. The nanoprobe according to claim 9, wherein the nanoprobe has a length of 1 μm to 5 μM.

22. A method of measuring pH in a single cell, comprising:
(a) inserting a nanoprobe into the single cell, wherein the nanoprobe is prepared by labeling a pH responsive fluorescent material to the surface of a nanowire grown on a tapered tip of an optical fiber;
(b) injecting a light through the optical fiber into the nanoprobe;
(c) exciting the pH-responsive fluorescent material by the light to generate fluorescence;
(d) transmitting the fluorescence signal generated from the fluorescence material according to pH in the cell, through the optical fiber; and
(e) analyzing the fluorescence signal to obtain a pH value in the cell.

23. The method of measuring pH in a single cell according to claim 22, wherein the fluorescence signal acquired through the optical fiber is transmitted to a spectrometer via an optical coupler.

24. The method of measuring pH in a single cell according to claim 22, wherein the measurement of pH value is obtained from spectral data of fluorescence in the spectrometer.

25. The method of measuring pH in a single cell according to claim 22, wherein the light incident through the optical fiber is laser, LED, near infrared, or visible light.

26. The method of measuring pH in a single cell according to claim 22, wherein the light incident through the optical fiber has a wavelength of 300 nm to 1000 nm.

27. The method of measuring pH in a single cell according to claim 22, wherein the light incident through the optical fiber has a wavelength of 400 nm to 700 nm.

28. An apparatus for measuring pH in a single cell, comprising:
a nanoprobe formed by labeling a pH-responsive fluorescent material to a nanowire grown on a tapered tip of an optical fiber;
a manipulator capable of regulating a three-dimensional movement of the nanoprobe so as to insert the nanoprobe into a single living cell;
a light source for applying light to the optical fiber;
an optical coupler for connecting the optical fiber with another optical fiber so as to transmit the light incident through the optical fiber to the nanoprobe and so as to transmit a fluorescence signal obtained from the nanoprobe through the another optical fiber; and
a spectrometer for obtaining a pH value by receiving the fluorescence signal through the another optical fiber and analyzing spectral data from the fluorescence signal.

29. A method of preparing a nanowire material solution according to claim 1, comprising steps of:
mixing a mixture of PVC (0.014 g, 131 mmol) and sodium azide (0.010 g, 220 mmol) in anhydrous DMF solvent (0.7 mL) in an amber vial at 70° C. and then covering the vial with an aluminum foil to block light;
adding methanol (0.5 mL) to the mixed solution after 2 hours of reaction, and centrifuging the same at 10,000 rpm for 1 minute to remove an excess unreacted reagent and precipitate an azide-functionalized polymer; and
drying the obtained precipitates in a vacuum condition for 1 hour and then dissolving the precipitates by adding an NMP solvent (50 μL).
